(19) United States
(12) Patent Application Publication
(10) Pub. No.: US 2007/0056052 A1
(43) Pub. Date: Mar. 8, 2007

(54) Methods of generating knock-out rodents

(76) Inventor: Michael N. Gould, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/493,280

(22) Filed: Jul. 26, 2006

Related U.S. Application Data
(62) Division of application No. 10/286,628, filed on Oct. 31, 2002.
(60) Provisional application No. 60/335,117, filed on Oct. 31, 2001.

(51) Int. Cl. A01K 67/027

(52) U.S. Cl. 800/14

(57) ABSTRACT

A method for generating knock-out rodents including rats and mice is disclosed. The method involves mutagenizing a rodent with a mutagen, obtaining progeny of the mutagenized rodent, and identifying, among the progeny, one progeny that carries a loss-of-function modification of a target gene. The preferred mutagen for generating knock-out mice and rats is N-ethyl-N-nitrosourea (ENU). The preferred screening assays for identifying a progeny of a mutagenized animal that carries a loss-of-function modification are yeast truncation assays and yeast functional assays. Knock-out rodents generated by the method of the present invention are also within the scope of the invention.

### CROSS-REFERENCE TO RELATED APPLICATIONS

This application is a divisional application of U.S. application Ser. No. 10/286,628 filed on Oct. 31, 2002, which claims the benefit of U.S. provisional application Ser. No. 60/335,117, filed on Oct. 31, 2001.

### STATEMENT REGARDING FEDERALLY SPONSORED RESEARCH OR DEVELOPMENT

This invention was made with United States government support awarded by the following agency: NIH grant numbers CA28954 and CA77494. The United States has certain rights to this invention.

### BACKGROUND OF THE INVENTION

An important tool for studying the function of a gene is to knock out the gene from an animal. In addition, many human diseases can be modeled by knocking out certain genes in animals. Furthermore, knocking out genes may produce traits in animals that are commercially valuable.

Currently, knock-out animals are usually generated using embryonic stem (ES) cells. However, the ES cell method works only in a few species such as mice. Even with mice, the ES cell method works only in a few strains. Despite the extensive efforts in the last ten years to use the ES cell method to create knock-out rats, no one has been able to do so successfully. The efforts to make the ES cell method work in more mouse strains only achieved very limited success. Furthermore, the ES method often has the problem of leaving residual exogenous DNA at the site of the knocked out gene.

Another approach that has been proposed to generate knock-out animals is the nuclear transfer method. Besides the problem of residual exogenous DNA left at the site of knocked out gene, the nuclear transfer method has the additional problem of epigenetic instability, which causes difficulty in determining whether a phenotype observed in a knock-out animal is purely a consequence of the absence of the target gene or is confounded by molecular developmental events related to the epigenetic instability. This problem is unlikely to be eliminated by back-crossing.

### BRIEF SUMMARY OF THE INVENTION

In one aspect, the present invention relates to a method of producing knock-out rodents. In one embodiment, the method involves mutagenizing a rodent with a mutagen, obtaining progeny of the mutagenized rodent, and identifying, among the progeny, one progeny that carries a loss-of-function modification of a target gene. A homozygous knock-out rodent for the target gene can be obtained through breeding.

In another embodiment, the method of the present invention involves mutagenizing ES cells of a mouse with a mutagen, screening the ES cells to identify one or more cells that carry a loss-of-function modification of a target gene by a biological screening assay, and recovering a knock-out mouse from an ES cell identified in the screening step. This ES cell method of the present invention differs from the known ES cell method in that a high efficiency biological screening assay is used.

The preferred mutagen for generating knock-out mice and rats with the method of the present invention is N-ethyl-N-nitrosourea (ENU). The preferred screening assays for identifying a progeny of a mutagenized rodent that carries a loss-of-function modification are yeast truncation assays and yeast functional assays.

Knock-out rodents generated by the method of the present invention are also within the scope of the invention.

In another aspect, the present invention relates to a knock-out rat that carries a loss-of-function modification in a pre-selected target gene in all of its germ cells and somatic cells. The loss-of-function modification is a result of manipulation of the genome of the knock-out rat or an ancestor of the knock-out rat. Therefore, the knock-out rat of the present invention does not include a naturally-occurring rat whose genome has not been manipulated by a human being.

It is an advantage of the present invention that the knock-out animals generated do not have the residual exogenous DNA problem (associated with the conventional ES cell technology and the nuclear transfer technology) and the epigenetic instability problem (associated with the nuclear transfer technology).

It is another advantage of the present invention that when the mutagenization and the identification of loss-of-function modification steps are optimized, creating knock-out animals by the method of the present invention is cost-efficient.

Other objects, features and advantages of the present invention will be apparent from the following detailed description when taken in conjunction with the accompanying claims and drawings.

### BRIEF DESCRIPTION OF THE SEVERAL VIEWS OF THE DRAWINGS

FIG. 1 is a schematic representation of the p53 functional assay. 1. Male rats are treated with the mutagen ENU. 2. Following a period of reduced fertility, ENU-treated rats are bred and their progeny are subjected to tail clippings from which total RNA is isolated. 3. p53 RNA is reverse-transcribed and amplified by PCR. 4. Unpurified PCR products are co-transformed into yeast with a linearized expression vector carrying the 5′ and 3′ ends of the p53 open reading frame (numbers indicate codons). Gap repair of the plasmid with the PCR products results in constitutive expression of the p53 protein. 5. Yeast cells that have repaired the plasmid through homologous recombination are selected on media lacking leucine. The yeast lack an endogenous ADE2 gene but have an exogenously added ADE2 open reading frame downstream of a CYC1 minimal promoter containing three copies of the RGC p53 binding site. The medium contains a low but sufficient amount of adenine, resulting in the formation of small red colonies for mutant p53 Ade cells, and large white colonies for wild-type p53 ADE⁺ cells.

FIG. 2 shows examples of universal vectors for DNA truncation assays. Two universal gap vectors were constructed. Both were built upon the pLSK870 backbone vector by inserting the universal gap repair linker at the Not I site with a unique Sma I restriction site to separate the 5′- and 3′- universal gap repair linker sequences. One of the universal gap repair linkers was adopted from the study of Kataoka et al.(Kataoka et al., 2001). The second universal gap repair linker was chosen by random selection from the genetic codon table, with the resulting linker sequence showing no homology to known genes. Hybrid primer pairs with gene-specific 3′-sequences and 5′-universal vector sequences were used to amplify specific cDNA or gDNA fragments. The flanking 5′-universal linker sequences from the hybrid primers can enable the amplified PCR products to be cloned into the matched linearized universal gap vector by homologous recombination after co-transfection into yeast.

FIG. 3 shows SD male rat sterility after ENU treatment. Male Sprague Dawley (SD) rats were given ENU as a single dose of 120 mg/kg body weight (hatched bars, n=6) or a split dose of 2×60 mg/kg at a one week interval (gray bars, n=5) or 0 mg/kg (black bars, n=4-6). These males were then bred to SD female rats for consecutive 2-3 week periods beginning 3 weeks post-ENU. The percentage of males able to produce viable litters is plotted versus the specific test period post-ENU administration.

FIG. 4 shows Brca1 and Brca2 yeast cDNA/gDNA truncation assays. Male rats are treated with ENU and bred to produce F1 pups. DNA and RNA are isolated from tail clips of one-week-old F1 rats. Total RNA is reverse-transcribed and both the resultant cDNA (Brca1) and isolated genomic DNA (Brca2) are amplified using PCR for selected DNA regions. The backbone vector was customized for each region by cloning in small 5′ and 3′ sequences from the fragment of interest. For Brca1, three vectors were generated and the third vector (used for the cDNA assay) is shown. The 5′ and 3′ sequences for this vector are derived from nucleotides 3974-4075 and 5464-5548 of the Brca1 cDNA (GenBank #AF036760), respectively. For Brca2, three vectors were also generated and the second is shown. The 5′ and 3′ sequences for this vector are derived from nucleotides 3518-3618 and 5101-5204 of the Brca2 cDNA (GenBank #U89653, mRNA), respectively. The vectors shown are those that ultimately led to the identification of the knock-outs. These 5′ and 3′ end sequences from each fragment were cloned in tandem and separated by a unique Sma I restriction enzyme site, which allows the plasmid to be linearized such that the target gene fragments are situated at the 5′ and 3′ ends of the linearized vector. The linearized vector is then co-transformed together with unpurified PCR product of either a Brca1 or a Brca2 fragment into competent yeast (S. cerevisiae, yIG397 strain) cells. Following transformation, the gene-specific fragment is cloned in vivo into the gap-repair vector by homologous recombination, which is almost fully efficient in yeast. Once incorporated into the vector, the Brca1 or Brca2 fragment is then located behind the efficient yeast promoter ADH1 and in front of the reporter gene ADE2, with which it jointly codes for a functional chimeric protein. This yeast strain lacks ADE2 function that can be restored by this chimeric protein. Yeast cells that produce chimeric ADE2 protein grow efficiently and form large white colonies when plated. In the absence of functional chimeric protein the yeast cells grow poorly and form small red colonies. Thus, if the DNA donor F 1 pup is wild-type for the incorporated gene fragment, the assay yields large white colonies. If, however, the donor rat DNA contains a functional mutation in one allele of Brca1 or Brca2 in the assayed fragment, the translation of a functional hybrid ADE2 protein is prevented and small red colonies are produced. In this assay, a functional mutation in a rat will be heterozygous; therefore, approximately half the colonies will be red and half white after accounting for a background rate of red colonies (about 15% from non-mutant plates for the cDNA assay, and about 1% from the gDNA assay).

FIG. 5 shows a loss-of-function mutation identified in a Brca2 knock-out rat. Yeast cells co-transformed with gap vector and a PCR product enriched for Brca2-fragment 2 (nucleotides 3518-5204) were plated on selective medium. When genomic DNA obtained from a rat (SD) with two wild-type alleles was assayed, the resultant plate contained mostly large colonies. In contrast, when the DNA is from a rat in which one allele of Brca2 was functionally mutated, the resultant colonies were an almost equal mixture of red and white colonies, which were picked and used to obtain Brca2-fragment 2 DNA sequence. The sequence of white yeast colonies (FIG. 5, upper, representative of 4 colonies tested) is that of wild-type rat Brca2, while the sequence of red colonies (FIG. 5, center, representative of 8 colonies tested) has a transversion mutation at T-4254 (indicated by the arrow) of the cDNA (TAT (tyrosine) to TAA (stop)). Genomic DNA from the heterozygous knock-out rat #3983 contains both T and A at nucleotide 4254 as seen in the lower sequence (represents 2 independent tests). The sequences shown in FIG. 5 span bases 4242-4266 of the rat Brca2 cDNA.

FIG. 6 shows a loss-of-function mutation identified in a Brca1 knock-out rat. Yeast cells were co-transformed with linearized gap vector and a PCR product enriched for Brca1 fragment 3 (nucleotides 3974-5548). A plate with 44.3% red colonies (with an average 15.8% red colony background from all other plates) identified a potential knock-out rat #5385. a) Sequence of haploid DNA from a yeast red colony (representative of 8 colonies tested) in which exon 22 (74 bp) is deleted when compared to the sequence of haploid DNA from a wild-type white colony (panel b, representative of 2 colonies tested). The arrow in panel a indicates the first nucleotide (5359) of exon 23, while the arrow in panel b indicates the first nucleotide (5285) of exon 22. This difference is highlighted by sequencing a mixture of cDNA from both rat alleles (+/−) from a RT reaction of total tail RNA (panel c, representative of 2 independent tests). In panels a, b, and c, the sequence prior to the arrow is the 3′ end of exon 21. Panel d shows the results of sequencing genomic DNA from a wild type SD rat over a region of intron 21 that contains the splicing branch site (underlined), while panel e shows this same sequence from the heterozygous Brca1 mutant founder rat #5385 which includes a T to C mutation (indicated by the arrow) within the splicing branch site. The sequences shown in panels d and e span from nucleotides 36-12 upstream of exon 22, with the mutation at nucleotide 24 upstream of exon 22.

FIG. 7 shows translation of Brca1 mutant transcript of a Brca1 knock-out rat. The Brca1 mutant mRNA from rat #5385 and wild-type mRNA sequences are shown from cDNA positions 5203-5571 with their translations shown below the DNA sequences. The positions of the exon borders are indicated by arrows, and the deletion of exon 22 in the Brca1 mutant is indicated by dashes. Codon ggg at the exon 21/23 junction is shown in bold, as is the glycine (G) amino acid encoded by it. This frameshift results in a premature stop codon (tga) at the exon 23/24 border (shown in bold and underlined). The wild-type stop codon (taa) is shown in the last position.

FIG. 8 is a schematic representation of the Agouti yeast cDNA truncation assay. A small piece of ventral skin from ACI or (SD×ACI) F 1 rats was excised and used for total RNA isolation and RT-PCR of the Agouti gene. A single gap vector was constructed by the same methods as for Brca1 and Brca2 using the 5′ and 3′ sequences derived from nucleotides 55-119 and 435-484 of the Agouti mRNA sequence (GenBank #AB045587), respectively. This vector was co-transformed together with unpurified RT-PCR product of the Agouti gene into competent yeast (S. cerevisiae, yIG397 strain) cells. The wild-type Agouti gene (e.g., from ACI rats) codes for a functional fusion protein with the ADE2 gene of the vector and forms large white colonies when plated. A truncated Agouti gene (e.g., from SD rat alleles) will not form a functional protein and the colonies will be small and red.

### DETAILED DESCRIPTION OF THE INVENTION

The present invention relates to a method for producing knock-out rodents. Examples of knock-out rodents that can be produced by the method of the present invention include but are not limited to rats and mice. The knock-out rodents produced by the method of present invention are also within the scope of the invention. As shown in Example 4 below, one strain of Brca1 and one strain of Brca2 knock-out rats have been successfully produced. With regard to knock-out mice, although the conventional ES method is available for certain strains, the method of the present invention can be used for more strains and has the advantage of not leaving residual exogenous DNA at the site of the knocked out gene.

The term “knock-out rodents” as used herein means rodents that are either heterozygotes or homozygotes with regard to a loss-of-function modification of a target gene. A loss-of-function modification of a gene means a mutation the end result of which is that no protein with the normal function of the wild-type gene product is made from the gene or only a protein with diminished function is made. The term “loss-of-function modification” is used interchangeably with the term “loss-of-function mutation” in the specification and claims.

The method of the present invention for producing knock-out rodents involves mutagenizing a rodent with a mutagen, obtaining progeny of the mutagenized rodent, and identifying, among the progeny, at least one progeny that carries a loss-of-function modification of the target gene. A variation of the method involves mutagenizing a rodent with a mutagen, obtaining progeny of the mutagenized rodent, collecting germ cells and preferably one or more other tissues as well from the progeny, identifying germ cells of a progeny that carry a loss-of-function modification of a target gene, and recovering a knock-out rodent from the germ cells identified. Germ cells of a progeny that carry a loss-of-function modification of a target gene can be identified by analyzing a polynucleotide sample prepared from a portion of the germ cells collected, or preferably, by analyzing a polynucleotide sample prepared from other tissues collected from the same progeny.

Any mutagen that is known to effectively produce mutations in a rodent of interest can be used. These mutagens, which include but are not limited to ENU, X-ray, y-rays, ethyl methane sulfonate (EMS), N-nitroso-N-methylurea (NMU) and N-methyl-N′-nitro-N-nitrosoguanidine (MNNG), are known to one of ordinary skill in the art. Preferably, a mutagen of very high efficacy for a rodent of interest is used to make the process more cost-efficient and less time-consuming. For example, ENU is a preferred mutagen for mice and this disclosure teaches that ENU is also a preferred mutagen for rats. The Example 3 below discloses preferred mutagenization strategies for outbred Sprague Dawley rats, inbred Fischer 344 rats and inbred Wistar-Furth rats using ENU. Mutagenization strategies for other rodents using ENU and mutagenization strategies for rodents using other mutagens can be similarly determined.

Rodents of either sex can be mutagenized. Preferably, a male rodent is mutagenized for progeny reproduction efficiency. In order to produce progeny of a mutagenized rodent, the mutagenized rodent can be mated with a wild-type rodent or another mutagenized rodent. Often times, after mutagen treatment, a rodent experiences a transient loss of reproductive capability, either partially or completely, after which the capability will be regained. It is preferable that the mutagenized rodents are bred to produce progeny after the reproduction capability is regained. However, they can be bred to produce progeny before and during the transient loss period if the loss is only partial. It should be noted that when ENU is used as a mutagen for rats, the rats that have lost reproductive capability completely may not regain it back (Example 3 below). The preferred progeny for the present invention are F1s due to cost and mutation efficiency concerns. Other progeny, such as F2s and other backcross progeny, are also acceptable.

There are many ways that a loss-of-function mutation of a target gene can be identified and all of them can be used in the present invention. For example, physical methods can be used. Physical methods are those that are based on analysis DNA or RNA sequences. Examples of physical methods include single strand conformation polymorphism (SSCP), denaturing HPLC (DHPLC) and direct sequencing (reviewed in Beier, D. R., Mamm. Genome, 11: 594-597, 2000). The physical methods currently available are relatively expensive on a per base level for large scale screens and thus not preferred methods for purpose of the present invention.

Another class of methods that can be used in the present invention for identifying loss-of-function mutations of target genes is called biological screening methods. Biological screening methods involve amplifying a suitable DNA or RNA sequence of a target gene and introducing the sequence into a reporter system for a biological function to which a loss-of-function mutation in the target gene can lead to a detectable change. There are many reporter systems that can be used in the present invention. For example, a reporter system can be a vector in a suitable host cell (e.g., yeast, bacteria or other cell lines) wherein the vector contains a promoter and a reporter gene between which a sequence of a target gene can inserted. In this system, certain loss-of-function mutations in the target gene can be detected when its insertion into the vector disrupts the normal expression of the reporter gene in the host cell.

As another example, if the target gene can regulate transcription through a regulation element, a reporter system can be a reporter vector in a suitable host cell (e.g., yeast, bacteria or other cell lines) wherein the vector contains a reporter gene the transcription of which is under the control of the element. In this system, the reporter vector is introduced into the host cell along with an expression vector carrying the target gene obtained from progeny of a mutagenized animal. Any loss-of-function mutations in the target gene can be detected based on a reduced or complete loss of expression of the reporter gene in the host cell.

Three specific biological screening methods that are preferred for purpose of the present invention are described in Example 1 below. These methods are termed cDNA truncation assay, gDNA truncation assay and functional assays. Although yeast are used as host cells and ADE2 gene is used as a reporter gene in these methods, a skilled artisan appreciates that other host cells and reporter genes can also be used. The functional assay can be used for genes whose functions are known and for which a functional test are available or can be designed. The two truncation assays can be used for any gene. For the functional assay and the cDNA truncation assay, any tissue that expresses the target gene can be used for the identification of a loss-of-function mutation. For the gDNA assay, all tissues can be used.

Once a progeny of a mutagenized rodent is identified as carrying the loss-of-function mutation of the gene of interest, the progeny can be used to generate a homozygous rodent for the loss-of-function mutation of the gene. Another way to obtain the above-mentioned homozygous rodent is to collect sperm, oocytes or embryos from progeny of a mutagenized rodent and identify which batch of sperm or oocytes carry the loss-of-function mutation of the gene of interest. Then, in vitro fertilization can be performed with the sperm or oocytes to generate heterozygous, and eventually homozygous, rodents for the loss-of-function mutation of the gene of interest.

It should be noted that when a high efficacy mutagen such as ENU is used in the method of the present invention, a knock-out rodent generated may carry a loss-of-function modification in some other genes in addition to the target gene. A knock-out rodent with regard only to the target gene can be obtained through back-crossing. The back-crossing method has been widely used in the art to generate congenic animals and a skilled artisan is familiar with the method.

The mutagenization and biological screening methods describe above can be used on ES cells to generate knock-out mice in strains in which the conventional ES cell method has been used successfully. First, mouse ES cells are mutagenized with a mutagen and then screened for cells that carry a loss-of-function modification of a target gene using a biological screening method. For example, single cell colonies can be formed from the mutagenized ES cells and a polynucleotide sample from each colony can be prepared for screening for loss-of-function modifications of a target gene. Preferred biological screening methods include yeast cDNA truncation assay, yeast gDNA truncation assay and yeast functional assays. Next, an ES cell that has been identified to carry a loss-of-function modification of the target gene is used to recover a knock-out mouse in the same way as that of the conventional ES cell method.

The invention will be more fully understood upon consideration of the following non-limiting examples.

### EXAMPLE 1

### Biological Mutation Screening Methods

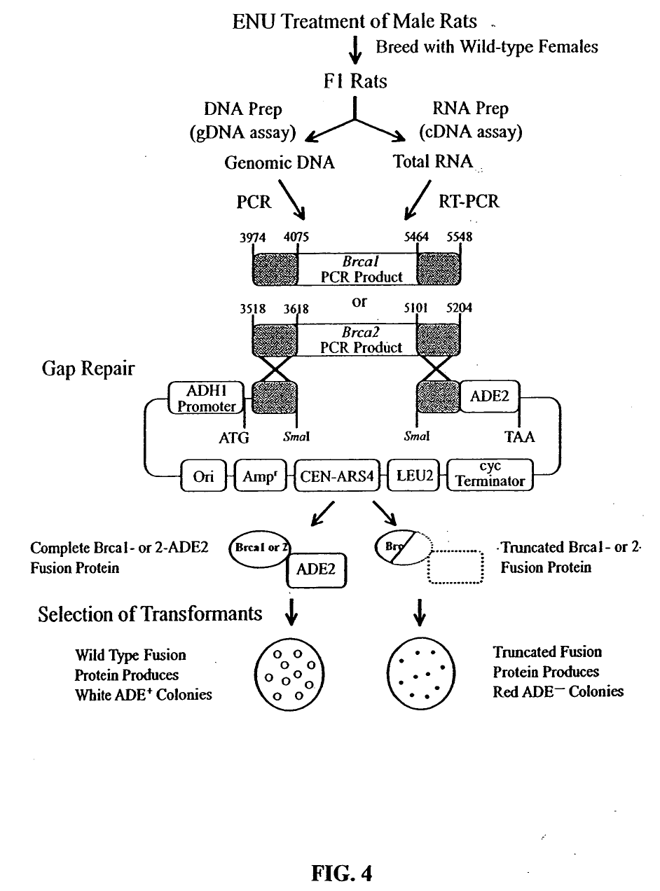

Yeast gDNA and cDNA truncation assays: These two assays can be best understood in view of FIG. 4, which shows a specific embodiment of the assays. The first step for detecting functionally mutated target genes with these two assays is to isolate total RNA or gDNA from progeny of mutagen-mutagenized rodents. For each gene one wishes to target for knock-out using RNA as a starting material (cDNA assay), one can design oligonucleotide primers for both reverse transcription (RT) and PCR. The gDNA assay uses genomic DNA as a template for PCR. If a gene's predicted cDNA is smaller than 2 Kb (the average gene is approximately 1.5 kb) or its largest exon is less than 2 Kb (for gDNA assay), only one primer set is needed for PCR. If it is greater than 2 Kb, one can divide the predicted cDNA or exonic genomic DNA into fragments so that each PCR product is generally 1.5-2 Kb. Next one can use these unpurified PCR-produced DNAs to transform yeast. For each gene or gene fragment one can engineer a specific yeast gap vector. A gap vector is one that, when linearized, is repaired by homologous recombination, allowing the rapid in vivo cloning of a cDNA (FIG. 4). For both truncation assays (cDNA, and gDNA) all target genes can use the same vector backbone and yeast strain. For example, the gap vector for the truncation assays is a plasmid containing the yeast ADH1 promoter driving the ADE2 gene (FIG. 4). One can insert a DNA cassette that combines a short sequence, from both the 5′ and 3′ ends of the cDNA or gDNA to be screened, between the promoter and ADE2. Each cassette has a unique restriction site between the 5′ and 3′ sequences. This allows vector linearization at the border of the 5′ and 3′ sequence. Yeast cells are co-transformed with the linearized gap vector and the total PCR products containing the selected DNA or DNA fragment. The yeast cells then perfectly insert the fragment in frame, as designed, between the promoter and ADE2 using homologous recombination, which is very close to 100% efficient. This results in a robust fusion protein between the chosen gene and ADE2 (FIG. 4). This fusion protein is termed robust because the ADE2 chimeric proteins have been shown to be functional with the great majority of its fusion partners. The plasmid vector now has a “control” function residing in the target gene sequence that, if mutated to yield a nonsense or out-of-frame frameshift mutation, prevents the translation of the ADE2 chimeric RNA. When ADE2 chimeric protein is expressed and functional, the yeast colony is white; when it is not, colonies are red and smaller in size. In summary, together, these two truncation assays should be able to detect mutations in any targeted gene; however, they will mainly detect mutations that inhibit accurate or efficient synthesis of the ADE2 chimeric protein (e.g., nonsense and most frameshift and deletion mutations).

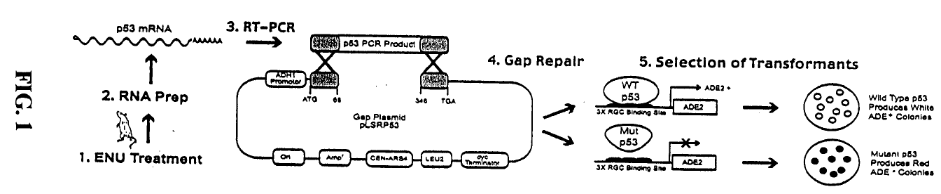

Yeast functional protein assays: FIG. 1 shows a specific embodiment of the yeast functional protein assays using p53 as an example. The functional protein yeast assay has both advantages and disadvantages when compared to the truncation assays. One advantage is that it has the potential to detect all classes of mutations that significantly modify protein function, including missense mutations. It also has the potential to screen for hypo- and hypermorphic mutations. It is, however, limited to genes with known functions that can be used to devise unique yeast assays. It is also generally dependent on using RNA as a starting material except for genes that lack introns. This assay is thus of a specialized nature in contrast to the versatility of the truncation assays. The creative challenge for the functional protein assay is the design of the specific “control” function related to the target gene. The functional protein assay is based on the loss-of-function of the transcribed protein. Thus, unlike the ease for the truncation assay, it is critical for the design of a functional protein assay that we have a good understanding of a protein's biological function. The functional protein assay should be capable of detecting the great majority of functional mutations including all missense mutations, in-frame frameshifting and deletions, as well as nonsense and out-of-frame mutations. However, since this assay and the cDNA truncation assay both use cellular RNA as a starting material they are potentially compromised. This is because of the cellular process of nonsense-mediated RNA decay (NMD). In mammalian cells, mRNAs containing a nonsense mutation that is not in the last exon will be subject to varying degrees of elimination at the ribosome during protein synthesis (Frischmeyer et al., 1999). However, as shown in Example 4 below, the yeast cDNA truncation assay used therein was able to detect a loss-of-function mutation for Brca1 even in the presence of NMD. In addition, the NMD problem may be minimized as has been demonstrated in cultured cells (Howard et al., 1996), human tissue samples (Andreutti-Zaugg et al., 1997), and in mice (Barton-Davis et al., 1999). Some specific methods for minimizing NMD are described below in Example 2. In contrast, the gDNA truncation assay will not be affected by NMD since the starting material is genomic DNA (rather than RNA). The major limitation of the gDNA assay will be determined by the size of the largest exon of a target gene. This assay is less cost efficient for genes in which no exon is larger than approximately 400-500 bp. The larger the exon the more cost efficient the assay.

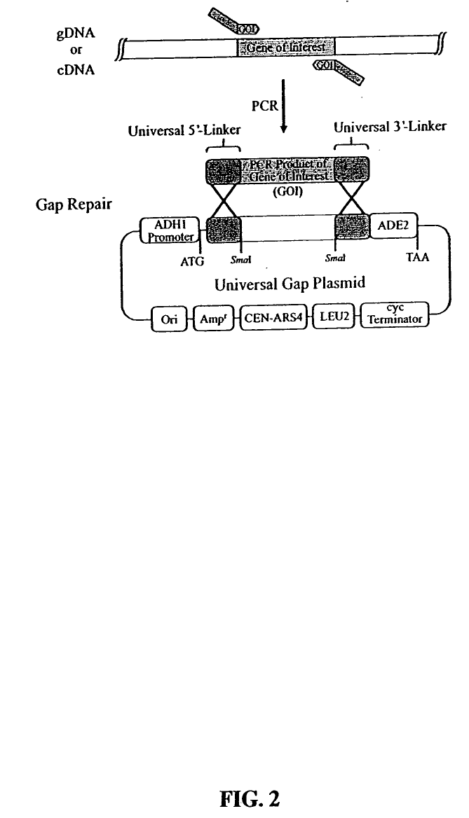

Development and evaluation of a universal truncation yeast vector: The yeast gap repair vectors shown in FIG. 4 and Example 4 incorporate specific sequences from the 5′ and 3′ ends of the target gene or target gene fragments. This approach works well but requires customizing a specific vector for each knock-out target. A universal vector (FIG. 2) can be developed and used in the yeast truncation assays. The universal gap repair vector of Kataoka et al. (Kataoka et al., 2001) utilizes the 3′ terminus of CYC promoter sequence and the 5′ terminus of ADE2 reporter gene as the universal repair sites. In addition to the sequences used in the study of Kataoka et al., other selected sequences may also be used. These artificial sequences will not match known gene sequences. As is the case for the gene-specific vectors, these new universal vectors have a defined restriction site between the 5′ and 3′ gap repair sequences so that when linearized the vector's ends will each have either the 5′ or 3′ universal gap repair sequence. In the yeast truncation assays described in FIG. 4 and example 4, the entire primer is based on target gene sequence. The primers used in conjunction with the universal vectors are hybrid, of which the 3′-half will continue to recognize the target gene sequence while the 5′-half is homologous to the universal vector gap repair sequence (FIG. 2). Gene-specific 3′-termini of paired hybrid primers enable the PCR amplification of a specific gene. The 5′-termini of paired hybrid primers will enable the amplified PCR product to be cloned into a matched universal vector in yeast by homologous recombination. The PCR primers and vector are designed to maintain the ORF of the target gene.

A specific example for creating a universal vector for the truncation assay: The oligonucleotides for the universal linker used by Kataoka et al. (Kataoka et al., 2001) with Not I overhangs are as follows: Upper primer, 5′-GGCCTACACACACTAAATTAATAATGACCCCCGGGATGGATTCTAGAACAGTTGGTA TAT (SEQ ID NO:1); Lower primer, 5′-GGCCATATACCAACTGTTCTAGAATCCATCCCGGGGGTCATTATTAATTTAGTGTGTG TA (SEQ ID NO:2). Oligonucleotides for the artificial gap linker are as follows: Upper primer 5′-GGCCATCGATAGCTCGATGTAACGTGCAGCCCGGGGTTAAGCATAGCGTATCTGTTA GTA (SEQ ID NO:3); Lower primer, 5′-GGCCTACTAACAGATACGCTATGCTTAACCCCGGGCTGCACGTTACATCGAGCTATC GAT (SEQ ID NO:4). The paired gap linker oligonucleotides were annealed to each other by heat-denaturing the oligonucleotide mixture in 10 mM Tris-HCl pH7.6, 25 mM NaCl, 1 mM EDTA buffer and cooling slowly at 4° C. The annealed universal gap linkers were cloned into our Not I-linearized pLSK870 backbone vector. Hybrid Brca2 primers with 18-base, 24-base or 30-base universal gap linker overhangs can be used to amplify Brca2 exon 11 genomic DNA fragments. The yeast truncation assays for the universal gap vectors can be carried out in the same manner as was done for the Brca1 and Brca2 gap vectors (Example 4 below).

### REFERENCES

Andreutti-Zaugg, C., Scott, R. J., and Iggo, R. Inhibition of nonsense-mediated messenger RNA decay in clinical samples facilitates detection of human MSH2 mutations with an in vivo fusion protein assay and conventional techniques. Cancer Res., 57: 3288-3293, 1997.
Barton-Davis, E. R., Cordier, L., Shoturma, D. I., Leland, S. E., and Sweeney, H. L. Aminoglycoside antibiotics restore dystrophin function to skeletal muscles of mdx mice. J. Clin. Invest., 104: 375-381, 1999.
Frischmeyer, P. A., and Dietz, H. C. Nonsense-mediated mRNA decay in health and disease. Human Mol. Genet., 8: 1893-1900, 1999.
Howard, M., Frizzell, R. A., and Bedwell, D. M. Aminoglycoside antibiotics restore CFTR function by overcoming premature stop mutations. Nat. Med., 2: 467-469, 1996.
Kataoka, A., Tada, M., Yano, M., Furuuchi, K., Cornain, S., Hamada, J.-I., Suzuki, G., Yamada, H., Todo, S., and Moriuchi, T. Development of a yeast stop codon assay readily and generally applicable to human genes. Am. J. Pathol., 159: 1239-1245, 2001.

### EXAMPLE 2

### Minimizing NMD and Other Methods for Improving Mutant Yield

Minimizing NMD: NMD can be reduced by treating rodents with NMD—minimizing drugs, which are familiar to a skilled artisan. Examples of such drugs include but are not limited to gentamicin and protein synthesis inhibitors. Gentamicin, which has a very low short-term toxicity profile even at high doses (Barton-Davis et al., 1999), facilitates the read-through over a stop codon during the translation process and thus interferes with NMD (Frischmeyer et al., 1999; Howard et al., 1996; Barton-Davis et al., 1999). This is similar to the action of suppressor tRNAs. Barton-Davis et al. showed the efficacy of gentamicin in minimizing NMD in vivo using the MDX mouse, a Duchenne Muscular Dystrophy model with a stop codon in the MDX gene (Barton-Davis et al., 1999). This drug strategy has also been shown to work in a cystic fibrosis model in vitro (Howard et al., 1996). In addition, protein synthesis inhibitors (e.g., emetine, cyclohexamide, or puromycin alone or in combinations with aminoglycosides) can also reduce NMD. For example, Iggo and colleagues showed the efficacy of puromycin for reducing NMD in human tissue samples such as WBC and the resultant improvement in the protein functional assay (Andreutti-Zaugg et al., 1997). The efficacy of cyclohexamide, which can safely be given to rats (Buchkremer-Ratzmann et al., 1996), has recently been demonstrated in that it increased mRNA levels of the mutated HEXA allele from undetectable to 40% of the wild-type allele (Rajavel et al., 2001). This has also been observed for a cell line with a CYP1A1 mutation in which the steady-state level of CYP1A1 RNA mutant allele was fully restored by both cyclohexamide and puromycin (Lei et al., 2001). Nomura et al. showed that the treatment of freshly drawn whole blood with puromycin suppressed the NMD of hMSH2 and hMLH1 (Nomura et al., 2000). All of the drugs mentioned above can be used independently or in combination with one another.

NMD can also be reduced using genetic methodologies. For example, a cDNA assay (3′ about 2000 bp of ORF) and a gDNA assay (last exon 25, about 1000 bp in the mouse), as well a functional protein yeast assay can be developed to target the Rent1 or Rent2 gene for knock-out. Rent1 and Rent2 are essential genes in NMD (Medghalchi et al., 2001; Mendell et al., 2000). Details of a functional protein assay are described below. Rent1 was recently knocked out in the mouse; however, it was embryonic lethal in homozygous null mice. In the viable heterozygous knock-outs, Rent1 RNA was reduced by 50% (Medghalchi et al., 2001). Even though this reduction did little by itself to minimize NMD in the test system used, rats heterozygous for Rent1 may interact in an additive or super-additive manner with NMD-minimizing drugs. Restoring Rent1 or Rent2 in rats with targeted knock-outs of other genes on a Rent1 or Rent2 heterozygous background can be readily accomplished with a single backcross. An alternative genetic strategy can also be used to produce transgenic rats with a dominant-negative Rent1 (Sun et al., 1998) driven by a universal mammalian promoter such as Rosa-21. This dominant-negative Rent1 gene carries an Arg to Ser mutation at aa 844 in the RNA-helicase domain. When stably expressed in HeLa cells it minimized the NMD of the β-globin gene carrying a nonsense mutation. This dominant-negative RENT1 raised the mutant allele RNA from 16% of total to 35% of total without affecting the level of the nonsense mutation-free allele (Sun et al., 1998). Again, such a transgenic rat can be given NMD inhibitor drugs in order to obtain an additive or super-additive effect. Alternatively, or in addition, it can be crossed with the Rent1 or Rent2 heterozygous knock-out rats to further titrate the levels of functional Rent1 or Rent2 and thus NMD.

A Functional Protein Assay for Rent1: A variation of the allosuppression assay that was used to discover the Upf1p gene in yeast as modified to test the functionality of Rent1 in yeast (Perlick et al., 1996) can be used as a functional assay for Rent1. Yeast strain PLY38 (MATa, ura3-52, his4-38, SUF1-1, upf1-2) can be used. This yeast strain has a +1 frameshift mutation in the HIS4 transcript near the 5′ end (his4-38). This results in both translation inhibition at an adjacent stop codon and a major decrease in mRNA for this gene via NMD. This yeast strain also carries SUF1-1 which encodes for a frameshift suppressor tRNA and lacks Upf1p function. This suppressor decodes the four base codon that contains the frameshifting base as glycine and allows a low level of translation through the +1 frameshift. This suppressor tRNA is temperature sensitive, working best at 30° C., and is inhibited at 37° C.-39° C. Thus at a normal temperature (30° C.) this yeast is able to grow well in media lacking histidine because of the absence of functional Upf1p and the presence of active suppressor tRNA. However at 37° C.-39° C. it grows poorly in this deficient medium because of the low activity of the suppressor tRNA at this temperature range (Perlick et al., 1996).

It has been shown that if this yeast is transformed with Upf1p it will not grow at 39° C. while it will grow slowly at 30° C. When RENT1 was used to transform yeast it did not complement the Upf1p-deficient phenotype. This is likely because while RENT1 conserves most of the known functional domains of Upf1p it lacks sequence homology at the 3′ and 5′ regions of the yeast gene. If a RENT1/Upf1p chimeric gene which encodes for Upf1p 5′ UTR and N-terminus (aa1-59), the functional region of RENT1 (aa121-917) and the Upf1p 3′ terminus (aa854-971) and the 3′ UTR is used to complement the Upf1p-deficient phenotype, a major inhibition of growth in histidine-free media was observed at 39° C. (Perlick et al., 1996).

A yeast gap vector which incorporates rat Rent1 as a chimeric protein with the above specified 3′ and 5′ regions of Upf1p gene can be designed. The gap vector's insert region contains the 5′ UTR and first 177 coding bases of Upf1p followed by a limited number (about 100 bp) of bases from the 5′ region of the functional body of Rent1. This is followed by a limited number (about 100 bp) of bases from the 3′ region of the body of Rent1. These two pieces of Rent1 are joined by a vector-unique restriction site (Sma I) to allow for its linearization. Following the Rent1 sequence are the coding bases for aa854-971 of Upf1p and its 3′ UTR. The sequence of the rat Rent1 gene can be determined by standard sequencing/cloning methods based on its high level of conservation between mice and humans (Mendell et al., 2000) or by searching the rat trace database (from the rat genome project) using the mouse Rent1 gene sequence. The functional body of Rent1 is located in aa121-917 which is encoded by 2388 nucleotides. This is at the upper border for efficient high cDNA yield from the RT/PCR portion for the assay. Alternatively, a slightly smaller region of about 2,000 bp can be used and the excluded approximately 388 bp between the Rent1 5′ and 3′ sequence bp can be added into the gap vector while maintaining the Sma I site.

The gap vector can be linearized and co-transformed with the Rent1-containing PCR product into yeast (PLY38 strain). Following homologous recombination, this chimeric protein is driven by the ADH1 yeast promoter. For each rat assay two plates of yeast can be grown: one at 30° C. and the other at 39° C. in histidine-deficient medium. If both of the rat Rent1 alleles (central region) are wild-type, a uniform difference in colony size will be seen between all the colonies in plates grown at 39° C. vs 30° C. (larger colonies at 30° C.). This temperature-dependent difference in growth will, however, be minimized in about 50% of the colonies grown at 39° C. if one Rent1 rat allele is functionally mutated and inactivated by ENU. In other words, at 39° C. approximately half the colonies will be larger than the other half since they incorporated the non-functional mutated transcript coded by the mutant rat allele. The faster growing yeast colonies at 39° C. result from inability to destroy mRNA for histidine by NMD since they lack both Upf1p activity and have their suppressor tRNA inactivated at 39° C.

Alternatively, a functional protein assay for Rent1 using color selection (red) instead of a colony size heterogeneity selection can be designed and used. A vector-encoded chimeric protein of histidine and ADE2 can be developed and yeast strains PLY38 can be converted to ADE2.

Other genes that can be targeted for knock-out to improve mutant yield in mutagen-treated animals: One such gene is the alkyl guanine alkyl-transferase gene (Agat), which encodes an enzyme that removes alkyl adducts, in an error-free manner, from the O⁶ position of guanine—a major ENU mutagenic adduct (Pegg et al., 2000). A cDNA truncation assay with modifications to reduce potential NMD can be used. This small protein's largest exon in mouse is only 207 bp, likely making it unsuitable for a rat gDNA assay. This knock-out rat will be more sensitive to both ENU-induced mutagenesis and killing; however, the ratio of mutations to cell killing will be increased. This is based on the results of Tong et al., who showed that the ratio of mutations vs. killing in cultured cells was increased when the Agat enzyme was inactivated. Mutations were reported to increase 3-5 fold while cell killing increased by only 1.8 fold (Tong et al., 1997).

### REFERENCES

Andreutti-Zaugg, C., Scott, R. J., and Iggo, R. Inhibition of nonsense-mediated messenger RNA decay in clinical samples facilitates detection of human MSH2 mutations with an in vivo fusion protein assay and conventional techniques. Cancer Res., 57: 3288-3293, 1997.
Barton-Davis, E. R., Cordier, L., Shoturma, D. I., Leland, S. E., and Sweeney, H. L. Aminoglycoside antibiotics restore dystrophin function to skeletal muscles of mdx mice. J. Clin. Invest., 104: 375-381, 1999.
Buchkremer-Ratzmann, I., and Witte, O. W. Systemically administered cycloheximide reduces inhibition in rat neocortical slice preparation. Brain Res., 743: 329-332, 1996.
Frischmeyer, P. A., and Dietz, H. C. Nonsense-mediated mRNA decay in health and disease. Human Mol. Genet., 8: 1893-1900, 1999.
Howard, M., Frizzell, R. A., and Bedwell, D. M. Aminoglycoside antibiotics restore CFTR function by overcoming premature stop mutations. Nat. Med., 2: 467-469, 1996.30.
Lei, X.-D., Chapman, B., and Hankinson, O. Loss of CYP1A1 messenger RNA expression due to nonsense-mediated decay. Mol. Pharmacol., 60: 388-393, 2001.
Medghalchi, S. M., Frischmeyer, P. A., Mendell, J. T., Kelly, A. G., Lawler, A. M., and Dietz, H. C. Rent1, a trans-effector of nonsense-mediated mRNA decay, is essential for mammalian embryonic viability. Hum. Mol. Genet., 10: 99-105, 2001.
Mendell, J. T., Medghalchi, S. M., Lake, R. G., Noensie, E. N., and Dietz, H. C. Novel Upf2p orthologues suggest a functional link between translation initiation and nonsense surveillance complexes. Mol. Cell. Biol., 20: 8944-8957, 2000.
Nomura, S., Sugano, K., Kashiwabara, H., Taniguchi, T., Fukayarna, N., Fujita, S., Akasu, T., Moriya, Y., Ohhigashi, S., Kakizoe, T., and Sekiya, T. Enhanced detection of deleterious and other germline mutations of hMSH2 and hMLH 1 in Japanese hereditary nonpolyposis colorectal cancer kindreds. Biochem. Biophys. Res. Commun., 271: 120-129, 2000.
Pegg, A. E. Repair of 06-alkylguanine by alkyltransferases. Mutat. Res., 462: 83-100, 2000.
Perlick, H. A., Medghalchi, S. M., Spencer, F. A., Kendzior, R. J., Jr., and Dietz, H. C. Mammalian orthologues of a yeast regulator of nonsense transcript stability. Proc. Natl. Acad. Sci., U.S.A., 93: 10928-10932, 1996.
Rajavel, K. S., and Neufeld, E. F. Nonsense-mediated decay of human HEXA mRNA. Mol. Cell. Biol., 21: 5512-5519, 2001.
Sun, X., Perlick, H. A., Dietz, H. C., and Maquat, L. E. A mutated human homologue to yeast Upf1 protein has a dominant-negative effect on the decay of nonsense-containing mRNAs in mammalian cells. Proc. Natl. Acad. Sci. U.S.A., 95: 10009-10014, 1998.
Tong, H. H., Park, J. H., Brady, T., Weghorst, C. M., and D'Ambrosio, S. M. Molecular characterization of mutations in the hprt gene of normal human skin keratinocytes treated with N-ethyl-N-nitrosourea: influence of 06-alkylguanine alkyltransferase. Environ. Mol. Mutagen., 29: 168-179, 1997.

### EXAMPLE 3

### ENU Mutagenesis in the Rat: Optimization of Dosage and Production of Phenodeviants

Abstract

Genome-wide mutagenesis protocols using N-ethyl-N-nitrosourea (ENU) were optimized in three rat strains: inbred Wistar-Furth (WF), inbred Fischer 344 (F344) and outbred Sprague Dawley (SD). Nine-week-old male rats were given either a single intraperitoneal injection of ENU or a split dose with injections spaced a week apart. Fertility in the mutagenized males was determined at various times post-ENU treatment up to 26 weeks. While none of the ENU doses used were toxic to the male rats, the strains differed in their sensitivity to ENU-induced permanent sterility in a dose dependent manner, with the WF strain being the most sensitive and the SD strain able to tolerate the highest doses. In all strains tested, ENU-treated male rats rarely recovered fertility after a period of sterility. Fertile SD mutagenized male rats were used to generate F1 offspring and phenotypic mutant pups (phenodeviants) were visually identified. Abnormalities of the eyes, tail, and growth were those most commonly observed in the SD F1 pups. A large-scale phenotype screen revealed an observed phenodeviant rate of 1 in 65 using a split dose protocol of 2×60 mg/kg body weight in SD male rats compared to a spontaneous phenotypic mutation rate of 1 in 283 SD pups. A subset of the phenodeviant F1 rats was tested for inheritance of the phenotypic mutation. Results showed that several of the mutations were heritable.

Materials and Methods

ENU mutagenesis rat protocol: Pathogen-free inbred Wistar Furth (WF), inbred Fischer-344 (F344) and outbred Sprague Dawley (SD) male and female rats were obtained from Harlan Sprague Dawley, Inc. (Indianapolis, Ind.). All rats were given Teklad Lab Blox chow (Harlan Teklad, Madison, Wis.), acidified water ad libitum, and were housed under a 12-h light/12-h dark cycle. After a one week acclimation period, nine-week-old male rats were administered ENU as a single intraperitoneal injection; for a split dose, rats were injected with ENU once at 9 weeks of age and again at 10 weeks of age. One gram of ENU (Sigma) was dissolved in 10 mls of 95% ethanol and then diluted with 90 mls of phosphate citrate buffer prior to injection. All injections of ENU were given in the morning hours. To characterize the effect of ENU treatment on fertility in the various rat strains, mutagenized males were paired with untreated females of the same strain for consecutive 2-3 week periods, beginning 3-5 weeks after the first ENU treatment and continuing a minimum of 26 weeks post-ENU. We observed female rats for vaginal plugs, gross pregnancy and date of birth/size of litters. For our phenotypic mutant (phenodeviant) rat screening experiments, ENU-treated SD male rats were bred with SD female rats and all F1 pups were visually checked for gross abnormalities in physical development or behavior as compared to littermates at least twice prior to weaning at approximately 21 days of age. A subset of the F1 phenodeviant rats identified was bred to SD rats to determine inheritance of the observed phenotypic mutation. Several of the heritable phenodeviant rat lines are currently being maintained and backcrossed to eliminate residual ENU-induced genetic changes not associated with the phenotypic mutation.

Results

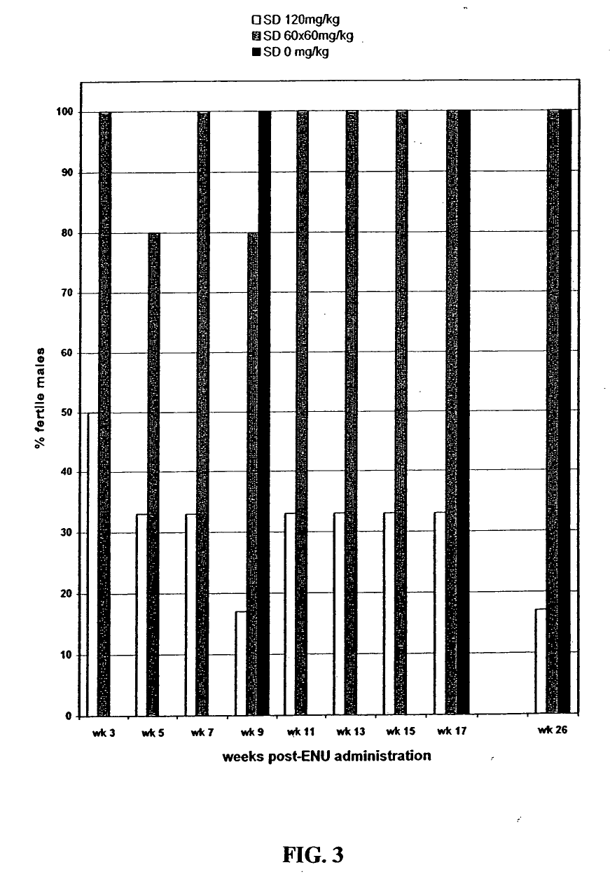

Optimization of ENU dosages: We examined several rat strains, inbred WF and F344 rats and outbred SD rats in which groups of male rats were given either single or split doses of ENU. These male rats were then bred to same-strain female rats at various times post-ENU to test for fertility (Table 1). The three rat strains were unable to regain fertility following large doses of ENU. The WF strain was extremely sensitive to ENU-induced sterility. Fertile WF male rats were obtained following a maximum total dose of 50 mg/kg ENU, but even at the lower doses tested, the majority of the treated WF males did not recover fertility. The SD and F344 strains responded in a similar manner with maximum ENU doses of 100-150 mg/kg, allowing for some percentage of rats to remain fertile or recover fertility; however, the F344 strain was more sensitive to split dose protocols compared to the SD rats. As shown in FIG. 3, ENU-treated SD rats rarely recovered fertility after a period of complete sterility, unlike many mouse strains. This phenomenon was also true for the both the F344 and WF rat strains. In fact, most of the fertile male rats remained fertile throughout the testing periods of weeks 3-26 following ENU treatment or showed brief reduced fertility periods (data not shown). It also appears from the SD and F344 fertility data shown in Table 1 that these rat strains were more likely to remain fertile or regain fertility following a split dose of ENU versus a single dose when the total dose exposure was equal. In addition, average litter size was reduced in both the SD and F344 strains around weeks 1-9 post-ENU (data not shown), the same time period in which we observed reduced fertility in the ENU-treated males. All fertile mutagenized male rats provided viable litters up to approximately one-year post-ENU treatment; however, as seen in mutagenized mice, the lifespan of these rats was shortened, with many developing skin and kidney tumors and lymphomas at approximately one year of age. None of the doses listed in Table 1 were acutely toxic to the rat strains tested.

TABLE 1
Effects of ENU treatment on male rat fertility^{a}

| | | | | Split dose | | |
|---|---|---|---|---|---|---|
| | Single dose | | | | | % |
| | dose in | % fertile | # fertile | dose in | % fertile | fertile |
| strain | mg/kg | males | males | mg/kg | males | males |
| SD | 75 | 100% | n = 3/3 | 2 × 50 | 100% | n = 6/6 |
| | 100 | 80% | n = 4/5 | 2 × 60 | 100% | n = 5/5 |
| | 120 | 33% | n = 2/6 | 2 × 75 | 20% | n = 1/5 |
| | 150 | 0% | n = 0/3 | 2 × 100 | 0% | n = 0/3 |
| | 200 | 0% | n = 0/3 | | | |
| | control | 100% | n = 5/5 | | | |
| F344 | 75 | 100% | n = 3/3 | 2 × 50 | 60% | n = 3/5 |
| | 100 | 67% | n = 4/6 | 2 × 60 | 40% | n = 2/5 |
| | 120 | 0% | n = 0/6 | 2 × 75 | 0% | n = 0/3 |
| | control | 100% | n = 6/6 | 2 × 100 | 0% | n = 0/3 |
| WF | 25 | 30% | n = 3/10 | 2 × 15 | 17% | n = 1/6 |
| | 35 | 33% | n = 2/6 | 2 × 25 | 17% | n = 1/6 |
| | 50 | 25% | n = 3/12 | 2 × 50 | 0% | n = 0/3 |
| | 75 | 0% | n = 0/3 | 2 × 75 | 0% | n = 0/8 |
| | 100 | 0% | n = 0/7 | | | |
| | control | 100% | n = 6/6 | | | |

^{a}ENU-treated male rats were paired with fertile female rats every two weeks from weeks 7-26 post-ENU administration. Vaginal plugs were observed for all infertile breeding pairs. Fertility was based upon ability to produce a viable litter when bred with females of the same strain.

Phenodeviant rat production: Multiple ENU dosing protocols in the SD rat strain determine their germline mutagenic potential by breeding ENU-treated SD males to females of the same strain to produce F1 offspring. These pups were examined for readily observable physical or behavioral abnormalities. Phenodeviants were identified at all doses listed in Table 2 with a variety of abnormalities observed. Phenodeviant F1 pups were born at various times between 6-52 weeks post-ENU treatment of male founders. The most frequent abnormalities visually identified were those of the eyes, tail, and growth. We expanded our phenodeviant rat production and screening using the split dose protocol of 2×60 mg/kg in the SD rat since preliminary results showed the greatest variety of phenodeviant F1 pups produced from this protocol. Selected phenodeviant rat F1 pups were maintained and bred to determine the heritability of the physical abnormalities (Table 3). For the single dose protocols, none of the six phenodeviant rats tested showed heritable mutations; however, several heritable phenotypic mutations were identified from rats generated from the split dose protocols. These abnormalities were diverse (Table 4).

TABLE 2
F1 Phenodeviant rats produced from ENU-treated SD males^{a}

| | SD | | | | |
|---|---|---|---|---|---|
| | single dose | | split dose | | |
| Abnormality observed | 100 | 120 | 2 × 50 | 2 × 60 | control |
| eye | 5 | 1 | 4 | 37 | 2 |
| tail | 0 | 1 | 3 | 13 | 1 |
| limb/digit | 0 | 0 | 0 | 5 | 0 |
| skin/hair | 0 | 0 | 0 | 6 | 0 |
| ear | 1 | 1 | 0 | 0 | 0 |
| craniofacial | 0 | 0 | 0 | 2 | 0 |
| gonad | 1 | 0 | 0 | 0 | 0 |
| behavioral | 1 | 0 | 0 | 0 | 0 |
| growth (<50% | 0 | 0 | 0 | 10 | 0 |
| littermates) | | | | | |
| multiple | 0 | 0 | 0 | 6 | 0 |
| total number of | 8 | 3 | 7 | 79 | 3 |
| phenodeviants | | | | | |
| total number of F1 rats | 1251 | 419 | 782 | 5163 | 849 |
| examined | | | | | |
| observed phenodeviant | 1 in 156 | 1 in 140 | 1 in 112 | 1 in 65 | 1 in 283 |
| rate | | | | | |

^{a}All F1 pups were visually examined for gross abnormalities in physical development or behavior at least twice prior to weaning at approximately 21 days of age.

TABLE 3
Determination of Heritable Phenotypes of F1 rats derived
from ENU-treated SD male rats

| Rat | ENU dose | # Phenodeviants | | non- | |
|---|---|---|---|---|---|
| Strain | mg/kg | observed | heritable | heritable | unknown^{a} |
| SD | 100 | 8 | 0 | 4 | 4 |
| SD | 120 | 3 | 0 | 2 | 1 |
| SD | 2 × 50 | 7 | 1 | 4 | 2 |
| SD | 2 × 60 | 79 | 9 | 9 | 61 |
| SD | 0 | 3 | 0 | 0 | 3 |

^{a}This group includes all phenodeviant F1 rats that were sterile or not evaluated, are currently being evaluated, or died prior to producing a litter.

TABLE 4
ENU-induced Heritable Phenotypes

| | F₁ | | | |
|---|---|---|---|---|
| | Founder | Initial ENU | | Confirmed in |
| Line | Sex | Dose (mg/kg) | Observed Phenotype | multiple litters |
| 18 | Female | 2 × 60 | crooked tail & slit eyes | yes |
| 19 | Male | 2 × 50 | growth on tail | yes |
| 28 | Female | 2 × 60 | red ring eyes | yes |
| 29 | Female | 2 × 60 | oblong face | yes |
| 32 | Female | 2 × 60 | slit eyes | yes |
| 38 | Male | 2 × 60 | curved tail | yes |
| 42 | Female | 2 × 60 | bald spots | yes |
| 60 | Female | 2 × 60 | scaly skin | no^{a} |
| 61 | Male | 2 × 60 | swollen feet | no^{a} |
| 63 | Male | 2 × 60 | additional digits on | yes |
| | | | hind feet | |

^{a}Only one litter has been produced to date; however, breeding of founder rat is ongoing.

From our studies, we have established protocols to efficiently mutagenize rat germ cells with ENU. Three rat strains were compared for their sensitivity to ENU using the endpoint of reproductive toxicity. It was observed that ENU could induce sterility in sexually mature male rats in a dose responsive manner. Interestingly, the rat, unlike the mouse, rarely recovered fertility following complete sterility. There was, however, a trend toward reduced sterility before of full fertility. We tested three rat strains to determine if differences existed between their sensitivity to ENU. All strains were tested with both a single dose and split dose administration protocol. The highest tolerated dose in each rat strain was defined as the maximum dose in which a percentage of the rats treated with ENU retained fertility by week 26 post-ENU treatment. It was found that these rat strains varied greatly in their tolerance to ENU, with the inbred WF rat as the most sensitive. Even following a very low dose of ENU at 25 mg/kg given in a single injection, approximately 70% of the male rats dosed were permanently sterilized. Similar sensitivities were observed with a split dose protocol. In contrast, the inbred F344 line was more resistant to ENU-induced sterility. All rats remained fertile at a dose of 75 mg/kg while at 100 mg/kg given in either a single or split dose, only about 60% of rats maintained fertility. While the maximum tolerated dose for the F344 strain was much higher than that for the WF strain, it was, however, lower than doses tolerated by many inbred mouse strains, i.e. 3×100 mg/kg is the recommended starting dose for mouse ENU mutagenesis experiments (Hrabé de Angelis et al. 2000; Balling 2001). In contrast, the outbred SD rats best tolerated ENU treatment. All SD rats given either a split dose of 2×50 mg/kg or 2×60 mg/kg were fully fertile following a brief recovery period. We chose to use the SD rat for further studies due to its tolerance to ENU treatment and its ability to produce large litters.

The number of F1 rats with deviant phenotypes derived from SD rats not treated with ENU and those dosed with single and split doses of ENU were quantified. We used a simple phenotypic mutant screen that consisted of visual observation of pups at various times prior to and at weaning of approximately 21 days of age. The results clearly showed that pups from ENU-treated males had a much greater frequency of abnormal phenotypes than did control rats. At a split dose of 2×60 mg/kg, 1 in 65 pups had readily detected visible phenotypic abnormalities. In comparison, offspring from non-treated SD males had similar abnormalities in only 1 in 283 pups. It was then determined if a subset of these F1 rats with physical abnormalities could pass on these traits to their offspring in a dominant manner. From the split dose protocols, we found heritable phenodeviant rats with traits varying from abnormal eyes, tails, skin, limbs and digits. These data suggest that ENU is an effective germline mutagen in the rat.

### REFERENCES

Balling R (2001) ENU mutagenesis: analyzing gene function in mice. Annu Rev Genomics Hum Genet 2, 463-492.
Hrabé de Angelis M, Flaswinkel H, Fuchs H, Rathkolb B, Soewarto D, et al. (2000) Genome-wide, large-scale production of mutant mice by ENU mutagenesis. Nat Genet 25, 444-447.

### EXAMPLE 4

### Production of knock-out rats using ENU mutagenesis and a yeast-based screening assay

Abstract

The rat is a widely used model in biomedical research and is often the preferred rodent model in many areas of physiological and pathobiological research. While many genetic tools are available for the rat, there is an important need for methods to produce gene-disrupted knock-out rats. In this study, we used N-ethyl-N-nitrosourea (ENU) to induce germ-line mutations in male Sprague Dawley (SD) rats. F1 pre-weanling pups from mutagenized male rats were then screened for functional mutations in Brca1 and Brca2 using a highly efficient, yeast gap-repair, ADE2-reporter truncation assay. Here we report the production of knock-out rats for each of these two breast cancer susceptibility genes.

Materials and Methods

Rat protocols: A split dose of ENU (2×60 mg/kg) was administered to male SD rats as described in Example 3. Mutagenized male rats were bred to untreated female SD rats to produce F 1 pups. Tail clips from the F 1 pups were collected at I week of age for macromolecule isolation. All breedings to produce ACI and (SD ×ACI) F1 pups were performed at our facility. At 3-7 days of age, all pups were sacrificed and ventral skin was collected for the Agouti yeast assay. All experimental animal procedures described in these studies have been approved by the University of Wisconsin-Madison Animal Care and Use Committee.

Vector Construction: The gap vector pLSRP53 containing the p53 cDNA (Flaman et al., 1995; Yamamoto et al., 1999) was digested with Hind III and Eag I to remove the entire p53 coding sequence. A 44-bp linker that contains sequence encoding the first 11 amino acids of rat p53 was inserted at the Hind III and Eag I sites to produce vector pLSK846 with the Eag I site converted to a unique Not I site. The full length ADE2 gene was PCR-amplified from yeast strain yIG397 (Flaman et al., 1995) DNA and integrated into the pLSK846 plasmid at the Not I site to generate vector pLSK870. A unique Not I site was retained at the 5′ end of the ADE2 gene. This Not I site was used to drop in Brca1, Brca2, or Agouti sequence cassettes. Each Brca1, Brca2, or Agouti cassette contained two fused approximate 100 bp fragments, corresponding to the 5′ and 3′ ends of an approximate 1.6 kb Brca1 fragment, an approximate 1.8 kb Brca2 fragment, or the approximate 500 bp Agouti ORF, joined by a unique Sma I site. The half-site sequences of the Brca1, Brca2, or Agouti cassettes were designed to be in frame with the p53 leader and ADE2 sequences (FIG. 4). Vectors were linearized before yeast transformation by digestion with Sma 1 (20 units/ml) followed by purification using a QIAquick PCR purification kit (Qiagen, Inc., Valencia, Calif.). For Brca1 and Brca2 positive controls, fragments 3 and 2, respectively, were mutated by site-directed mutagenesis to generate an in-frame stop codon and were then each cloned into a plasmid. The plasmids were used as template for PCR to generate mutant positive control fragments to be used in the yeast assay to yield roughly 99% red colonies.

DNA/RNA extraction: To isolate DNA, small sections of tails were digested overnight at 55° C. in 500 μl of genomic lysis buffer consisting of 20 mM Tris HCl pH 8.0, 150 mM NaCl, 100 mM EDTA and 1% SDS. Two hundred μl of Protein Precipitation Solution (Gentra Systems, Inc., Minneapolis, Minn.) was added to the lysate solution. DNA in the clear supernatant was precipitated with isopropanol, washed, and resuspended in water. Total RNA was isolated from tail or skin sections that were placed in RNAzol B solution (Tel-Test, Inc., Friendswood, Tex.) and homogenized (Polytron PT10⁻³⁵). The samples were then extracted with chloroform, precipitated with isopropanol, and washed with ethanol. Pellets were resuspended in 30 μl RNA suspension solution (Ambion, Austin, Tex.) for Brca1 and Brca2, and in 60 μl for Agouti.

RT and PCR: All primers used are listed in Table 5. cDNA was synthesized for Brca1 or Brca2 from 1-2.5 μg rat tail total RNA at 42° C. for 2 hours with 200 units of SuperScript II (Invitrogen, Carlsbad, Calif.). Agouti cDNA was synthesized from 1-5 μg of skin total RNA in a 1 hour reaction. The 20 PI reaction consisted of 1×RT buffer (Invitrogen), 0.5×RNA secure reagent (Ambion), 10 mM DTT, 1.25 mM dNTP mix, and 0.33 μg Brca1-, Brca2-, or Agouti-specific primers. PCR was performed on 1.0 μl of the cDNA product or about 1.0 μg of genomic DNA with 1 unit of Herculase (Stratagene, La Jolla, Calif.) in 20 μl reactions containing 1×Herculase Buffer, 0.2 mM dNTP mix and 0.05 μg primers for Brca1 and Brca2. Reaction conditions for Brca1 and Brca2 fragments were 95° C. for 2 min followed by 35 cycles consisting of 1 min at 92° C., 45 sec at 60° C., and 4 min at 72° C., followed by 7 min at 72° C. For the Agouti PCR, 0.5 units of Failsafe enzyme (Epicentre Technologies, Madison, Wis.) was used with Failsafe buffer J (which contains dNTPs) and 0.1 μg primers. The cycling conditions for Agouti were similar to above except that the annealing temperature was 55° C. and the 72° C. extension step is only 1 min. PCR quality and product quantity was verified by electrophoresis in a 1.2% agarose gel.

Yeast Transformation and Sequencing: yIG397 (Andreutti-Zaugg et al., 1997) yeast was cultured overnight at 30° C. in YPD medium supplemented with adenine (200 μg/ml) to an OD₆₀₀ of 0.9. The cells were washed and resuspended in a volume of LiOAc/TE solution (0.1 M lithium acetate, 10 mM trisHCl, pH 8.0, 1 mM EDTA) equivalent to the volume of the cell pellet. For each transformation, 30 μl of yeast suspension was mixed with 10 ng of linearized gap vector, 25 μg of salmon sperm carrier DNA, 150 μl of LiOAc/TE/PEG solution (0.1 M lithium acetate, 10 mM tris HCl, pH 8.0, 1 mM EDTA, 40% PEG) and 2-5 μl unpurified Brca1, Brca2, or Agouti PCR product (total volume about 185 μl). The mixture was incubated for 30 min at 30° C., then heat-shocked for 15 min at 42° C. Transformants were then plated on synthetic minimal medium lacking leucine and supplemented with low adenine (5 μg/ml) and incubated for 3 days at 30° C. For each group of samples, a positive control with the mutated PCR fragment amplified from the control plasmid and a negative control with vector and no PCR product were always run as well. An automated colony counter (ProtoCOL, Microbiology International, Bethesda, Md. USA) was used to determine the number of red and white colonies on each plate and the percentage of red colonies per sample was recorded. The background rate of red colonies was determined by averaging the % red colonies from all plates not containing a knock-out.

For sequencing, red and white colonies were picked directly into PCR mix amplified and purified to remove primers and nucleotides. Four μl of each reaction was then used in a 20 μl cycle-sequencing reaction using BigDye (Applied Biosystems Inc., Foster City, Calif.) chemistry. Since the PCR products for the Brca fragments are about 1600-1800 bp, we used 4 different sequencing primers spaced at about 500-600 bp intervals for Brca1 and 3 primers for Brca2, and only 1 primer for the approximate 500 bp Agouti fragment (Table 5).

TABLE 5

Primers used for RT, PCR, and sequencing

| Gene or | | | | |
|---|---|---|---|---|
| Fragment | | | Primer sequence, | |
| Tested | Template used | Primer name | 5′ to 3′ direction | Seq ID No. |
| RT Primers | | | | |
| Brca1 | mRNA | rBrca1-RT-P2/906^{a} | TGC ACG TTT GCA ACA | SEQ ID NO: 5 |
| Brca1 | mRNA | rBrca1-RT-P3/4124^{a} | CAG AGA GGT TTG CTT | SEQ ID NO: 6 |
| Brca1 | mRNA | rBrca1-RT-P4/5579^{a} | GAC GGG AAG ACC ATT | SEQ ID NO: 7 |
| Brca2 | mRNA | rBrca2-RT-P2/2195^{b} | GTG GCT TTT CTT CAC | SEQ ID NO: 8 |
| Brca2 | mRNA | rBrca2-RT-P3/7016^{b} | TGA ACA ATC GTC TGT | SEQ ID NO: 9 |
| Brca2 | mRNA | rBrca2-RT-P4/10219^{b} | ATT CCT GTC TGG ACA | SEQ ID NO: 10 |
| Agouti | mRNA | Agouti-RT(644) | CCC ACA ACT CAC AAC | SEQ ID NO: 11 |
| | | | CAC TG | |

PCR Primer Sets

| | | | | |
|---|---|---|---|---|
| Brca1, | gDNA | Brca1-F1-FP(850) | AAC CCC ACT GAG AAT | SEQ ID NO: 12 |
| fragment 1 | | | CAT GC | |
| | | Brca1-F1-RP(2696) | GCA AAT GAC TGA CGC | SEQ ID NO: 13 |
| | | | TTT GA | |
| Brca1, | gDNA | Brca1-F2-FP(2323) | AAA GAA CTC GGG GAT | SEQ ID NO: 14 |
| fragment 2 | | | TTG GT | |
| | | Brca1-F2-RP(4075) | CTT GAT CGC TAG CCT | SEQ ID NO: 15 |
| | | | CTT CC | |
| Brca1, | cDNA | Brca1-F3-FP(3973) | AGG CGT CAC CAG GCT | SEQ ID NO: 16 |
| fragment 3 | | | GAG AAT | |
| | | Brca1-F3-RP(5548) | ATC ATT GGA GTC TTG | SEQ ID NO: 17 |
| | | | TGG CTC | |
| Brca2, | gDNA and cDNA | Brca2-F2-FP(3435) | TCA TAA CTT AAC GCC | SEQ ID NO: 18 |
| fragment 2 | | | CAG CC | |
| | | Brca2-F2-RP(5267) | AAG GCA TTT CCT GCA | SEQ ID NO: 19 |
| | | | AAA TC | |
| Brca2, | gDNA | Brca2-F3-FP(4822) | TCA AAA CCC CTG AAG | SEQ ID NO: 20 |
| fragment 3 | | | GAC AG | |
| | | Brca2-F3-RP(6897) | ACC AGC CAT TCC TCC | SEQ ID NO: 21 |
| | | | TCT TT | |
| Agouti, | cDNA | Agouti-FP(53) | TGA AAC CTC CAG GAA | SEQ ID NO: 22 |
| whole ORF | | | CCA AG | |
| | | Agouti-RP(489) | ATC AGC AGT TGG GGT | SEQ ID NO: 23 |
| | | | TGA GT | |
| Gap vector | Yeast cells | yADH1-FP1 | CTG CAC AAT ATT TCA | SEQ ID NO: 24 |
| inserts | from colony, | | AGC | |
| | DNA not | yADE2-RP2 | CAT CAC ATT TTT CAG | SEQ ID NO: 25 |
| | extracted | | CTA GTT TTT C | |

Sequencing Primers

| | | | | |
|---|---|---|---|---|
| Brca1, | RT reaction | Brca1-F3-FP(3973) | AGG CGT CAC CAG GCT | SEQ ID NO: 26 |
| fragment 3 | products | | GAG AAT | |
| Brca1, | RT reaction | Brca1-F3-seq 4411 | GACAAATCCCAACCAC | SEQ ID NO: 27 |
| fragment 3 | products, | | AACC | |
| | Yeast colony | | | |
| | PCR products | | | |
| Brca1, | RT reaction | Brca1-F3-seq 4815 | TGCTGGTGGTGCTGATA | SEQ ID NO: 28 |
| fragment 3 | products, | | CTG | |
| | Yeast colony | | | |
| | PCR products | | | |
| Brca1, | RT reaction | Brca1-F3-seq 5218 | TCCCAGGAAAAGCTCTT | SEQ ID NO: 29 |
| fragment 3 | products, | | TGA | |
| | Yeast colony | | | |
| | PCR products | | | |
| Brca2, | RT reaction | Brca2-F2-FP(3435) | TCA TAA CTT AAC GCC | SEQ ID NO: 30 |
| fragment 2 | products | | CAG CC | |
| Brca2, | RT reaction | Brca2-seq 3997 | AGT AAG TGC CAG GTA | SEQ ID NO: 31 |
| fragment 2 | products, | | ACA GTA | |
| | Yeast colony | | | |
| | PCR products | | | |
| Brca2, | RT reaction | Brca2-seq 4612 | CAT TTC CCA ATT GGA | SEQ ID NO: 32 |
| fragment 2 | products, | | ACT GTC | |
| | Yeast colony | | | |
| | PCR products | | | |
| Gap vector | Yeast colony | yADH1-FP1 | CTG CAC AAT ATT TCA | SEQ ID NO: 33 |
| inserts | PCR products | | AGC | |

^{a}Three primers were pooled and used for the Brca1 RT reaction.
^{b}Three primers were pooled and used for the Brca2 RT reaction.

Results

Development of a yeast-based assay for mutation screening: Based on studies that established ENU-induced germ-line mutagenesis protocols for several rat strains, we chose to use the outbred Sprague Dawley (SD) rat for these studies due to its tolerance to ENU treatment, the variety of ENU-induced heritable phenotypic mutants identified, and large litter sizes (Example 3). We used a split dose of ENU (2×60 mg/kg) to mutagenize male SD rats. These rats were then bred to wild-type female SD rats to produce F1 pups that were screened for knock-out alleles of Brca1 and Brca2.

Two related truncation assays (Ishioka et al., 1993; Kataoka et al., 2001) were developed to screen the Brca1 and Brca2 genes of these F1 pups for functional mutations that could interfere with protein translation. The first assay termed the genomic DNA (gDNA) assay uses genomic DNA as a starting macromolecule while the second assay termed the cDNA assay begins with total RNA that is reverse transcribed (RT) to cDNA. Both the gDNA and cDNA truncation assays use their respective DNAs for the PCR amplification of fragments of a gDNA exon or fragments of the cDNA targeted for knock-out (FIG. 4). The unpurified total PCR product is co-transformed together with its corresponding linearized gap repair vector (FIG. 4) into yeast where the fragment is inserted into the plasmid by homologous recombination that is about 100% efficient. The gap repair vectors are customized for each targeted fragment.

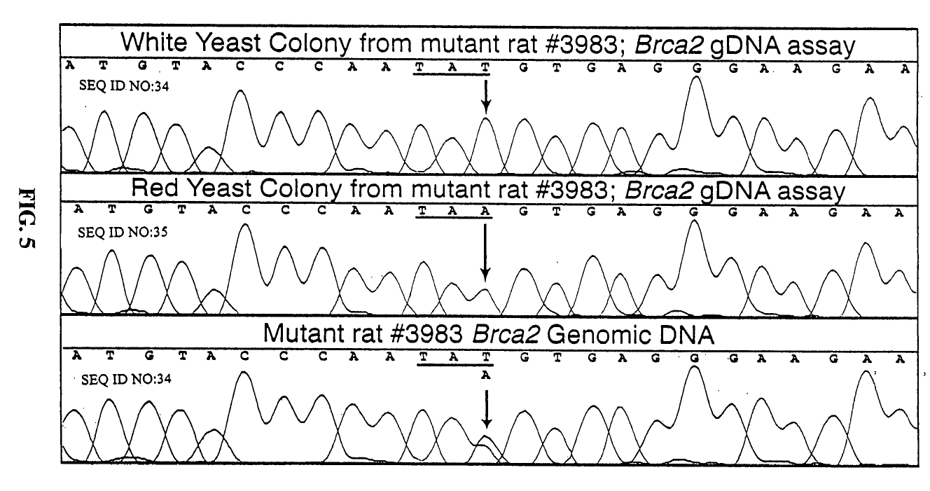

Establishment of a Brca2 knock-out rat line: We chose to first target the Brca2 gene, focusing on exon 11 (the largest exon, comprising roughly half of the cDNA) using a gDNA yeast gap repair truncation assay. This large exon was divided into three regions of about 1,800 bp each with some overlap across the ends of the fragments, and the second and third fragments were used for screening. Primer sequences used to amplify each fragment are shown in Table 5. For each Brca2 exon 11 region, a specific yeast gap vector was constructed from a universal backbone vector (FIG. 4). We screened genomic DNA from 1131 pre-weanling F1 rat pups before finding a mutated Brca2 allele using the second fragment/vector as shown in FIG. 4. The Brca2 knock-out rat was detected in our assay by a yeast plate that had approximately 45% red colonies and 55% white colonies. This initial assay was repeated and confirmed using an independent DNA sample from the founder rat 3983. Next, individual red and white yeast colonies were sequenced. Since these yeast are haploid, mutations are readily detectable. A nonsense transversion mutation was detected at nucleotide T-4254 of the Brca2 cDNA that converted TAT (tyrosine) to TAA (stop codon) at Tyr-1359 (FIG. 5, upper and center sequences). A/T to T/A transversion mutations are the most common mutation type (44%) found in ENU-induced germ-line phenotypic mutant mice (Justice et al., 1999; Noveroske et al., 2000). Genomic DNA from the founder rat 3983 was sequenced over the region of interest (see Table 5 for primers used) and was found to contain the identical mutation as detected in the yeast red colonies (FIG. 5, lower sequence).

The cDNA yeast assay was used in conjunction with the gDNA assay using the same Brca2 fragment 2 vector to screen N₂ pups resulting from the breeding of the Brca2 knock-out founder male rat 3983 to SD females. Both methods identified the same 9 out of 14 pups from the first litter of rats carrying this Brca2 mutation, and these results were confirmed by the direct sequencing of genomic DNA from each N₂ pup. This verified the utility of this yeast assay starting from either genomic DNA or RNA. The first six litters produced a total of 35 knock-outs out of 64 N₂ pups demonstrating the Mendelian inheritance of this knock-out gene in N₂ pups.

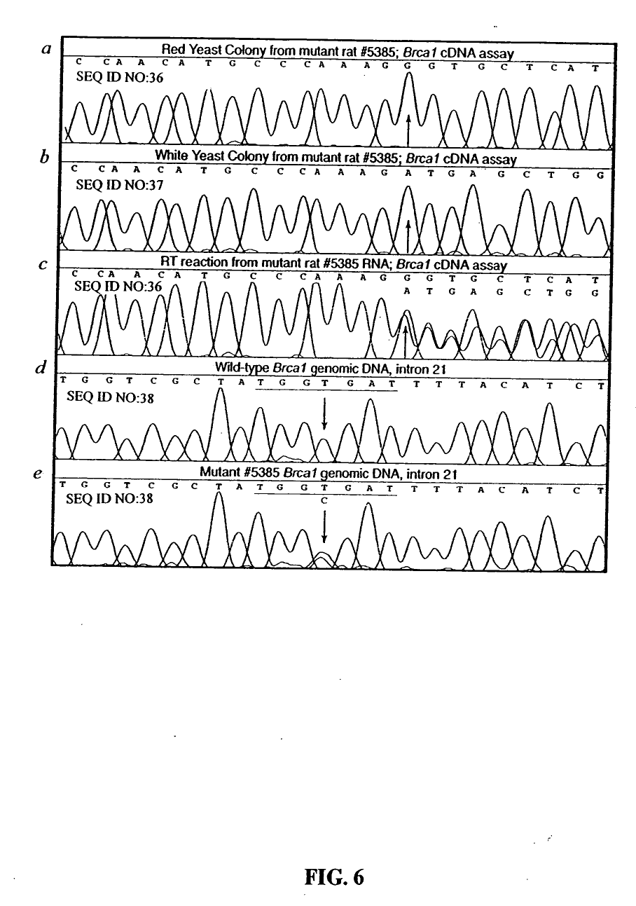

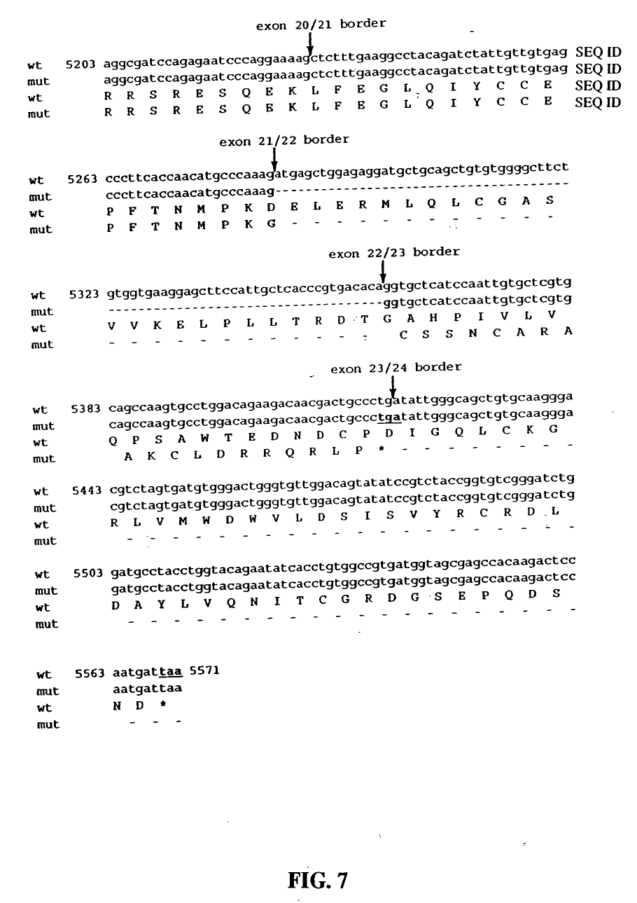

Production of a Brca1 knock-out rat: Customized gap repair vectors were prepared for screening of Brca1 (FIG. 4). These consisted of two gDNA vectors targeting exon 11 (the largest exon, target fragments 1 and 2) and one cDNA vector targeting Brca1 from the 3′ end of exon 11 to the end of the open reading frame (fragment 3). Table 5 lists the primer sequences for the three fragments. Following the screening of 2533 pups we identified a Brca1 knock-out in founder rat 5385 using the cDNA assay with the specific vector shown in FIG. 4. Haploid DNA from red yeast colonies was sequenced, revealing a complete loss of Brca1 exon 22 (74 bp) (FIG. 6a). We sequenced introns 21 and 22 in search of a splicing mutation to explain the loss of this exon. A T to C mutation was identified within the splicing branch site (TGGTGAT to TGGCGAT) (FIG. 6d, e). A T/A to G/C transition mutation is the second most common (38%) of ENU-induced mutations (Justice et al., 1999; Noveroske et al., 2000). The mutation in the branch site of intron 21 caused the splice donor site to skip over exon 22 and find a branch site in intron 22. This led to the splicing-out of the 74 bp exon 22 and also caused a frame shift downstream from exon 21 (FIG. 7) exposing a stop codon at the exon 23-24 border.

Nonsense-mediated decay: An anticipated problem using RNA as a starting material for this assay is the potential destruction of mRNA encoded by the mutant allele by cell surveillance mechanisms such as nonsense-mediated decay (NMD) (Culbertson et al., 1999; Frischmeyer et al., 1999; Kuramoto et al., 2001). NMD varies widely in its efficiency based on the specific gene and location of the mutation within the gene. We quantified the extent of NMD of the mutated Brca2 mRNA by comparing the yield of red colonies in the knock-out rat samples minus background in the wild-type samples using the cDNA assay (48.5%-15.3%) versus the yield of red colonies in the knock-outs minus background using the gDNA assay (44.8%-0.9%). The same gDNA Brca2-fragment 2 gap vector was used for both the cDNA and gDNA assays. From these results, NMD is calculated to occur at an approximate rate of [1−(33/44)] or 25%.

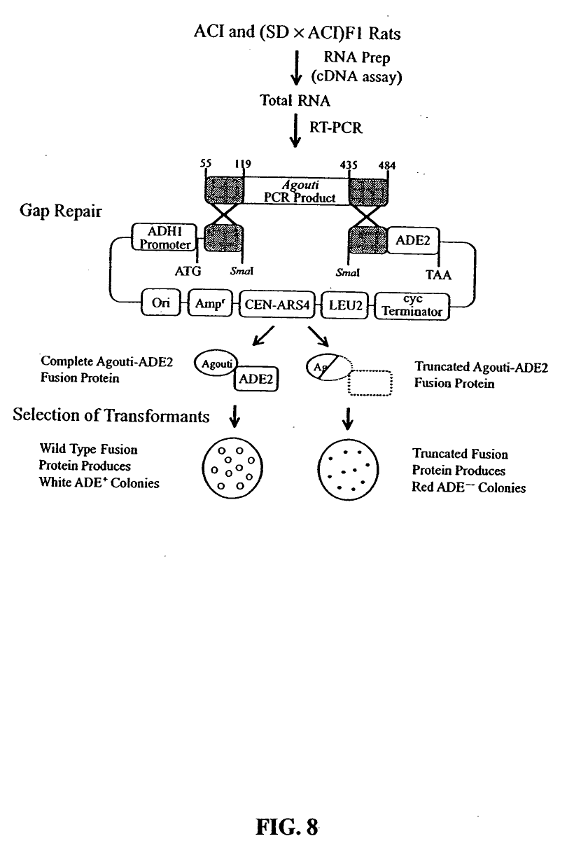

Since this level of NMD was modest we challenged our cDNA-based assay using a rat Agouti locus model in which approximately 85% of the mutant RNA is subject to NMD (Kuramoto et al., 2001). Agouti rat strains such as the ACI rat carry two copies of the wild-type locus and non-agouti rats such as Brown Norway (BN), SD, and F344 carry two identical, mutant alleles, each with two truncating mutations in the Agouti gene. We designed a yeast gap vector for this gene that allowed the entire ORF of the gene to be cloned in vivo in yeast (FIG. 8 and Table 5). Mutant alleles were assayed using the same strategy and methods used to assay Brca1 and Brca2 cDNA. We found that our assay could routinely detect the Agouti mutation in (SD×ACI) F1 pups, which had 15% red colonies, while the wild-type ACI group had 8% red colonies (background). NMD was estimated to remove 86% of the RNA from the mutated allele of the F1 pup, which corresponds well with the above-referenced ACI versus BN northern analysis (Kuramoto et al., 2001).

Discussion

Methods have been established to produce knock-out rats, and knock-outs for Brca1 and Brca2 have been identified. The technology used combined protocols for efficient germ-line mutagenesis by ENU and a method to economically and rapidly screen pre-weanling F1 rat pups from mutagenized fathers for functional mutations in selected genes using yeast truncation assays.

We present two versions of our yeast-based truncation screening assays that differ only in the starting macromolecule. The gDNA assay that was used to screen for the Brca2 knock-out started with genomic DNA while the cDNA assay used to screen for the Brca1 knock-out started with RNA that was converted to cDNA. Both yeast truncation assays have advantages and disadvantages that help suggest which one should be used in targeting each specific gene. The gDNA assay is most efficient if the selected gene has at least one exon larger than about 400-500 bp. In contrast, the RNA-based cDNA assay is independent of exon size and can easily incorporate up to about 2500 bp per vector. These truncation assays allow screening only for mutations that compromise protein translation such as nonsense mutations and out-of-frame frameshift deletions or insertions. The Brca1 knock-out rat was identified using a cDNA yeast truncation assay in the 3′ region of the Brca1 gene that consists of a series of very small exons. None of the exons covered would have been good targets for the gDNA truncation assay because of their small size. In addition, this knock-out would not have been found using other methods, such as sequencing, heteroduplex analysis, denaturing HPLC since these assays only screen exons from genomic DNA.

The major drawbacks to using the RNA-based cDNA assay are that the gene-specific RNA may not be produced in an easily collectable tissue and mutant RNA may be lost to a great extent by NMD. In these studies, we demonstrate the ability of a cDNA yeast-based screening assay to detect the Agouti mutant allele despite a high level of NMD in this model and the general ability of a yeast-based screening assay to detect mutants in spite of extensive NMD. NMD can be minimized by pre-treating collected cells, such as white blood cells, with a protein synthesis inhibitor before RNA collection. This approach has been successful for the yeast gap repair p53 assay (Flaman et al., 1995; Andreutti-Zaugg et al., 1997) and may be extrapolated to in vivo studies by the administration of a protein synthesis inhibitor to rat pups prior to tissue collection. We have had preliminary success in inhibiting NMD using the protein synthesis inhibitor emetine. The problem of a gene-specific RNA not being produced in tail tissue may be reduced by extending the range of biopsy tissues collected from viable rats, e.g., WBC, liver, skin, etc. In the future as it becomes possible to cryopreserve rat sperm from F1 male rats of mutagenized fathers, their sperm can be frozen and a wide variety of organ-specific RNAs could also be collected and stored, along with DNA from tails or spleens. DNAs or RNAs from a large number of rats could be screened and the appropriate frozen sperm used for the mutant rat recovery via in vitro fertilization and implantation. While sperm freezing is not yet available for the rat, it has been established for many mouse strains and crosses (Critser et al., 2000; Nakagata et al., 2000) and has allowed the recovery of a mutant mouse (Coghill et al., 2002).

Finally, this technology used to produce knock-out rats could easily be used in other species including the mouse. It could be used for mouse strains in which an ES cell approach has not been established. This technology is also more cost effective and rapid, requires less equipment and fewer specific skills than the ES cell technology, and does not leave residual exogenous DNA in the genome of the knock-out animal.

### REFERENCES

Andreutti-Zaugg, C., Scott, R. J. & Iggo, R. Inhibition of nonsense-mediated messenger RNA decay in clinical samples facilitates detection of human MSH2 mutations with an in vivo fusion protein assay and conventional techniques. Cancer Res. 57, 3288-3293 (1997).
Coghill, E. L. et al. A gene-driven approach to the identification of ENU mutants in the mouse. Nat. Genet. 30, 255-256 (2002).
Critser, J. K. & Mobraaten, L. E. Cryopreservation of murine spermatozoa. ILAR J. 41, 197-206 (2000).
Culbertson, M. R. RNA surveillance. Unforeseen consequences for gene expression, inherited genetic disorders and cancer. Trends Genet. 15, 74-80 (1999).
Flaman, J.-M. et al. A simple p53 functional assay for screening cell lines, blood, and tumors. Proc. Natl. Acad. Sci. USA 92, 3963-3967 (1995).
Frischmeyer, P. A. & Dietz, H. C. Nonsense-mediated mRNA decay in health and disease. Hum. Mol. Genet. 8, 1893-1900 (1999).
Ishioka, C. et al. Screening patients for heterozygous p53 mutations using a functional assay in yeast. Nat. Genet. 5, 124-129 (1993).
Justice, M. J., Noveroske, J. K., Weber, J. S., Zheng, B. & Bradley, A. Mouse ENU mutagenesis. Hum. Mol. Genet. 8, 1955-1963 (1999).
Kataoka, A. et al. Development of a yeast stop codon assay readily and generally applicable to human genes. Am. J. Pathol. 159, 1239-1245 (2001).
Kuramoto, T., Nomoto, T., Sugimura, T. & Ushijima, T. Cloning of the rat agouti gene and identification of the rat nonagouti mutation. Mamm. Genome 12, 469-471 (2001).
Noveroske, J. K., Weber, J. S. & Justice M. J. The mutagenic action of N-ethyl-N-nitrosourea in the mouse. Mamm. Genome 11, 478-483 (2000).
Nakagata, N. Cryopreservation of mouse spermatozoa. Mamm. Genome 11, 572-576 (2000).
Yamamoto, K. et al. A functional and quantitative mutational analysis of p53 mutations in yeast indicates strand biases and different roles of mutations in DMBA- and BBN-induced tumors in rats. Int. J. Cancer 83, 700-705 (1999).

The present invention is not intended to be limited to the foregoing examples, but encompasses all such modifications and variations as come within the scope of the appended claims.

I claim:

1. A Brca2 knock-out rat comprising a loss of function modification of its brca2 gene in all of its germ cells and somatic cells wherein the loss of function modification is introduced into said rat, or an ancestor of said rat through genome manipulation.

2. A Brca2 knock-out rat according to claim 1, wherein the Brca2 knock-out rat is rat number 3983.

3. A method for producing a Brca2 knock-out rat of claim 1 comprising the steps of:
mutagenizing a rat with a mutagen;
obtaining progeny of the mutagenized rat; and
identifying, among the progeny, one progeny that carries a loss-of-function modification of brca2.

4. The method of claim 3, wherein identifying a progeny that carries a loss-of-function modification is achieved by a biological screening method.

5. The method of claim 4, wherein identifying a progeny that carries a loss-of-function modification is achieved by a yeast truncation assay.

6. The method of claim 5, wherein the yeast truncation assay is a gDNA assay.

7. The method of claim 5, wherein the yeast truncation assay is a cDNA assay.

8. The method of claim 4, wherein identifying a progeny that carries a loss-of-function modification is achieved by a yeast functional assay.

9. The method of claim 3, wherein the mutagen is N-ethyl-N-nitrosourea (ENU).

10. The method of claim 3, wherein a male rat is mutagenized.

11. The method of claim 3, wherein a female rat is mutagenized.


SEQUENCE LISTING (42 sequences)

SEQ ID NO: 1 - DNA, 60 bp, Artificial Sequence
  Description of Artificial Sequencesynthetic oligonucleotide
ggcctacacacactaaattaataatgacccccgggatggattctagaacagttggtatat

SEQ ID NO: 2 - DNA, 60 bp, Artificial Sequence
  Description of Artificial Sequencesynthetic oligonucleotide
ggccatataccaactgttctagaatccatcccgggggtcattattaatttagtgtgtgta

SEQ ID NO: 3 - DNA, 60 bp, Artificial Sequence
  Description of Artificial Sequencesynthetic oligonucleotide
ggccatcgatagctcgatgtaacgtgcagcccggggttaagcatagcgtatctgttagta

SEQ ID NO: 4 - DNA, 60 bp, Artificial Sequence
  Description of Artificial Sequencesynthetic oligonucleotide
ggcctactaacagatacgctatgcttaaccccgggctgcacgttacatcgagctatcgat

SEQ ID NO: 5 - DNA, 15 bp, Artificial Sequence
  Description of Artificial Sequencesynthetic primer
tgcacgtttgcaaca

SEQ ID NO: 6 - DNA, 15 bp, Artificial Sequence
  Description of Artificial Sequencesynthetic primer
cagagaggtttgctt

SEQ ID NO: 7 - DNA, 15 bp, Artificial Sequence
  Description of Artificial Sequencesynthetic primer
gacgggaagaccatt

SEQ ID NO: 8 - DNA, 15 bp, Artificial Sequence
  Description of Artificial Sequencesynthetic primer
gtggcttttcttcac

SEQ ID NO: 9 - DNA, 15 bp, Artificial Sequence
  Description of Artificial Sequencesynthetic primer
tgaacaatcgtctgt

SEQ ID NO: 10 - DNA, 15 bp, Artificial Sequence
  Description of Artificial Sequencesynthetic primer
attcctgtctggaca

SEQ ID NO: 11 - DNA, 20 bp, Artificial Sequence
  Description of Artificial Sequencesynthetic primer
cccacaactcacaaccactg

SEQ ID NO: 12 - DNA, 20 bp, Artificial Sequence
  Description of Artificial Sequencesynthetic primer
aaccccactgagaatcatgc

SEQ ID NO: 13 - DNA, 20 bp, Artificial Sequence
  Description of Artificial Sequencesynthetic primer
gcaaatgactgacgctttga

SEQ ID NO: 14 - DNA, 20 bp, Artificial Sequence
  Description of Artificial Sequencesynthetic primer
aaagaactcggggatttggt

SEQ ID NO: 15 - DNA, 20 bp, Artificial Sequence
  Description of Artificial Sequencesynthetic primer
cttgatcgctagcctcttcc

SEQ ID NO: 16 - DNA, 21 bp, Artificial Sequence
  Description of Artificial Sequencesynthetic primer
aggcgtcaccaggctgagaat

SEQ ID NO: 17 - DNA, 21 bp, Artificial Sequence
  Description of Artificial Sequencesynthetic primer
atcattggagtcttgtggctc

SEQ ID NO: 18 - DNA, 20 bp, Artificial Sequence
  Description of Artificial Sequencesynthetic primer
tcataacttaacgcccagcc

SEQ ID NO: 19 - DNA, 20 bp, Artificial Sequence
  Description of Artificial Sequencesynthetic primer
aaggcatttcctgcaaaatc

SEQ ID NO: 20 - DNA, 20 bp, Artificial Sequence
  Description of Artificial Sequencesynthetic primer
tcaaaacccctgaaggacag

SEQ ID NO: 21 - DNA, 20 bp, Artificial Sequence
  Description of Artificial Sequencesynthetic primer
accagccattcctcctcttt

SEQ ID NO: 22 - DNA, 20 bp, Artificial Sequence
  Description of Artificial Sequencesynthetic primer
tgaaacctccaggaaccaag

SEQ ID NO: 23 - DNA, 20 bp, Artificial Sequence
  Description of Artificial Sequencesynthetic primer
atcagcagttggggttgagt

SEQ ID NO: 24 - DNA, 18 bp, Artificial Sequence
  Description of Artificial Sequencesynthetic primer
ctgcacaatatttcaagc

SEQ ID NO: 25 - DNA, 25 bp, Artificial Sequence
  Description of Artificial Sequencesynthetic primer
catcacatttttcagctagtttttc

SEQ ID NO: 26 - DNA, 21 bp, Artificial Sequence
  Description of Artificial Sequencesynthetic primer
aggcgtcaccaggctgagaat

SEQ ID NO: 27 - DNA, 20 bp, Artificial Sequence
  Description of Artificial Sequencesynthetic primer
gacaaatcccaaccacaacc

SEQ ID NO: 28 - DNA, 20 bp, Artificial Sequence
  Description of Artificial Sequencesynthetic primer
tgctggtggtgctgatactg

SEQ ID NO: 29 - DNA, 20 bp, Artificial Sequence
  Description of Artificial Sequencesynthetic primer
tcccaggaaaagctctttga

SEQ ID NO: 30 - DNA, 20 bp, Artificial Sequence
  Description of Artificial Sequencesynthetic primer
tcataacttaacgcccagcc

SEQ ID NO: 31 - DNA, 21 bp, Artificial Sequence
  Description of Artificial Sequencesynthetic primer
agtaagtgccaggtaacagta

SEQ ID NO: 32 - DNA, 21 bp, Artificial Sequence
  Description of Artificial Sequencesynthetic primer
catttcccaattggaactgtc

SEQ ID NO: 33 - DNA, 18 bp, Artificial Sequence
  Description of Artificial Sequencesynthetic primer
ctgcacaatatttcaagc

SEQ ID NO: 34 - DNA, 25 bp, rat
  misc_feature (1)..(25): bases 4242 to 4266 of wild type Brca2 cDNA
atgtacccaatatgtgagggaagaa

SEQ ID NO: 35 - DNA, 25 bp, rat
  misc_feature (1)..(25): bases 4242 to 4266 of mutant Brca2 cDNA
atgtacccaataagtgagggaagaa

SEQ ID NO: 36 - DNA, 24 bp, rat
  misc_feature (1)..(24): mutant Brca1 cDNA fragment
ccaacatgcccaaagggtgctcat

SEQ ID NO: 37 - DNA, 24 bp, rat
  misc_feature (1)..(24): wild type Brca1 cDNA fragment
ccaacatgcccaaagatgagctgg

SEQ ID NO: 38 - DNA, 25 bp, rat
  misc_feature (1)..(25): wild type Brca1 rat gDNA fragment
tggtcgctatggtgattttacatct

SEQ ID NO: 39 - DNA, 369 bp, rat
  CDS (1)..(369): bases 5203 to 5571 of wild type Brca1 cDNA
aggcgatccagagaatcccaggaaaagctctttgaaggcctacagatctattgttgtgagcccttcaccaacatgcccaaagatgagctggagaggatgctgcagctgtgtggggcttctgtggtgaaggagcttccattgctcacccgtgacacaggtgctcatccaattgtgctcgtgcagccaagtgcctggacagaagacaacgactgccctgatattgggcagctgtgcaagggacgtctagtgatgtgggactgggtgttggacagtatatccgtctaccggtgtcgggatctggatgcctacctggtacagaatatcacctgtggccgtgatggtagcgagccacaagactccaatgattaa

SEQ ID NO: 40 - protein, 122 aa, rat
RRSRESQEKLFEGLQIYCCEPFTNMPKDELERMLQLCGASVVKELPLLTRDTGAHPIVLVQPSAWTEDNDCPDIGQLCKGRLVMWDWVLDSISVYRCRDLDAYLVQNITCGRDGSEPQDSND

SEQ ID NO: 41 - DNA, 295 bp, rat
  CDS (1)..(144)
  misc_feature (1)..(295): mutant Brca1 cDNA fragment
aggcgatccagagaatcccaggaaaagctctttgaaggcctacagatctattgttgtgagcccttcaccaacatgcccaaagggtgctcatccaattgtgctcgtgcagccaagtgcctggacagaagacaacgactgccctgatattgggcagctgtgcaagggacgtctagtgatgtgggactgggtgttggacagtatatccgtctaccggtgtcgggatctggatgcctacctggtacagaatatcacctgtggccgtgatggtagcgagccacaagactccaatgattaa

SEQ ID NO: 42 - protein, 47 aa, rat
RRSRESQEKLFEGLQIYCCEPFTNMPKGCSSNCARAAKCLDRRQRLP